(19) United States
(12) Patent Application Publication
(10) Pub. No.: US 2025/0352462 A1
(43) Pub. Date: Nov. 20, 2025

(54) NOVEL COSMETIC OR DERMATOLOGICAL COMPOSITION CONTAINING A STRAIN OF LIVE PROBIOTIC BACTERIA

(71) Applicants: BASF Beauty Care Solutions France SAS, Lyon (FR); BASF Corporation, Florham Park, NJ (US)

(72) Inventors: Sabrina LEOTY-OKOMBI, Lyon (FR); Toan Van PHO, Tarrytown, NY (US); Corinne THIEL, Lyon (FR)

(21) Appl. No.: 18/874,650

(22) PCT Filed: Jun. 15, 2023
(86) PCT No.: PCT/EP2023/066044

§ 371 Date: Dec. 13, 2024

(30) Foreign Application Priority Data
Jun. 15, 2022 (FR) FR2205828

(51) Int. Cl. A61K 8/99; A61K 9/00; A61K 35/744; A61Q 19/00

(52) U.S. Cl. CPC A61K 8/99; A61K 9/0014; A61K 35/744; A61Q 19/00

(57) ABSTRACT

The invention relates to a cosmetic or dermatological composition comprising at least one live probiotic bacterial strain and at least one linear-chain lipodispersible and liposoluble molecule chosen from dicaprylyl carbonate, coco-caprylate, coco-caprate, undecane and/or tridecane, linoleic acid, behenyl alcohol or any one of their mixtures, in which the total content of the linear-chain lipodispersible and liposoluble molecules is at least 40% by weight, with respect to the total weight of the composition. The invention also relates to the use of a linear-chain lipodispersible and liposoluble molecule chosen from dicaprylyl carbonate, coco-caprylate, coco-caprate, undecane and/or tridecane, linoleic acid, behenyl alcohol or any one of their mixtures, for maintaining the viability of a live probiotic bacterial strain in a composition intended for administration topically.

### TECHNICAL FIELD

The invention belongs to the field of dermatological or cosmetic compositions containing a probiotic bacterial strain and at least 40% by weight of at least one linear-chain liposoluble and lipodispersible molecule and to their uses.

### Technological Background

It is recognized that probiotics are useful in topical application for the skin. Various clinical studies thus report the beneficial effects of probiotics on the skin in the cosmetic but also dermatological field.

Probiotics are indeed of advantage as cosmetic ingredients (M. Rahmati Roudsari et al., Health Effects of Probiotics on the Skin, Crit. Rev. Food. Sci. Nutr., 2015, 55(9), 1219-401. The use of probiotics can also have great potential in the prevention and treatment of skin diseases, in particular eczema, atopic dermatitis, acne and allergic inflammation. Other applications are mentioned, such as the treatment of skin hypersensitivity, UV-induced skin lesions and wound protection.

However, there are few probiotic products for topical applications available on the market due to technical difficulties related to the nature of this type of product. The main difficulty is to maintain the viability of the probiotic in the composition while preventing its proliferation, for the duration of manufacture but also of storage of the composition. This is because the majority of conventional cosmetic and/or dermatological formulations induce a loss of viability of the probiotic during manufacture and/or during storage, in particular due to the presence of compounds harmful to or biocidal for bacteria. Conversely, in the absence of these compounds, in particular in aqueous formulations, the probiotic can proliferate, making it impossible to preserve it.

Furthermore, topical compositions must make possible sufficient retention of the probiotic on the skin over time, which conventional formulations do not make possible [Garima Sharma, Manuhaar Sharma, Rishav Sood, Jayanthi Neelamraju, Suvarna G. Lakshmi, Ratna Sudha Madempudi, Praveen Rishi and Indu Pal Kaur (2021), Self-Preserving Gelatin Emulgel Containing Whole Cell Probiotic for Topical Use: Preclinical Safety, Efficacy, and Germination Studies, Expert Opinion on Drug Delivery, 18, 11, 1777-17891.

These difficulties thus hinder the use of probiotics by topical application, particularly in the cosmetic and dermatological fields.

The present invention is thus targeted at solving this technical problem by providing a composition which makes it possible to supplement the skin with probiotics by topical application in a safe, effective and practical manner while making it possible for the probiotic thus formulated to improve the health and/or the beauty of the skin locally.

Solutions already exist in these fields but exhibit disadvantages, such as excessively severe formulation or application constraints, and are insufficient. Thus, solutions of application by microneedles, patches or encapsulation of probiotics have been described (WO2020127637A1; Hui-Jiuan Chen et al., Transdermal Delivery of Living and Biofunctional Probiotics through Dissolvable Microneedle Patches, ACS Applied Bio Materials, 2018, 1 (2), 374-381)). Oily gels comprising an oil and an oily viscosity agent have also been described (WO2020/249734A1).

### SUMMARY OF THE INVENTION

In this context and unexpectedly and surprisingly, the Applicant Company has discovered that a composition intended for topical application, in particular cosmetic and/or dermatological application, containing at least 40% by weight of linear-chain liposoluble and lipodispersible molecules, at least one of which is chosen from specifically selected linear-chain liposoluble and lipodispersible molecules, optionally in combination with one or more other linear-chain liposoluble and lipodispersible molecules, makes it possible to solve this technical problem in a safe, simple, reliable, practical and efficient manner.

This is because the Applicant Company has discovered that the use of one or more specifically selected linear-chain liposoluble and lipodispersible molecules, optionally in combination with one or more other linear-chain liposoluble and lipodispersible molecules, at a total minimum content of linear-chain liposoluble and lipodispersible molecules of at least 40% by weight, with respect to the total weight of a composition intended for topical application, such as a cosmetic and/or dermatological composition, makes it possible to maintain the viability of a live probiotic bacterium and thus to solve this technical problem.

The specifically selected linear-chain liposoluble and lipodispersible molecules according to the invention are chosen from:
dicaprylyl carbonate,
coco-caprylate, optionally combined with coco-caprate, preferentially a mixture of coco-caprylate and of coco-caprate,
undecane and/or tridecane,
linoleic acid and/or oleic acid, and/or their linear-chain esters (e.g. isostearyl linoleate) and/or oleyl alcohol,
behenyl alcohol and/or its acid and ester derivatives, in particular behenic acid, otherwise denoted docosanoic acid, or docosanoate or behenate esters, and/or any one of their mixtures.

Linear-chain liposoluble and lipodispersible molecules are conventionally used in cosmetic and dermatological compositions for their emollient properties. However, it was not known that these molecules, and in particular those selected, when present at the minimum content of 40% by weight, with respect to the total weight of the cosmetic and/or dermatological composition, made it possible to maintain the viability of live bacteria contained in this composition and to thus solve the problems of the prior art, unlike other liposoluble and lipodispersible molecules, in particular branched molecules, as described in the prior art. This is because the examples show that branched-chain fatty substances, in the absence of the linear-chain liposoluble and lipodispersible molecules selected, do not make it possible to satisfactorily maintain the viability of bacteria.

The present invention thus makes it possible to develop topically applicable cosmetic and dermatological compositions which provide stability in a particularly advantageous manner, in particular long-term viability of the probiotic without proliferation of the latter for a satisfactory period of time compatible with the distribution and storage constraints in the cosmetic and dermatological fields, both for manufacturers but also for users. Thus, in these compositions, the probiotic bacterial strain is stable, that is to say that the strain remains alive and does not grow excessively and therefore remains stable until its use.

As is demonstrated in the examples, such compositions according to the invention remain stable, and have in particular maintenance of the viability and thus preservation of the probiotic bacterial strain which they contain advantageously up to:
at least 3 months at ambient temperature, that is to say at a temperature of between 18 and 25° C., in particular approximately 21° C., and
up to sixteen months, indeed even up to 18 months, at 4° C.

The linear-chain liposoluble and lipodispersible molecules used are of oily nature or waxes and thus make it possible to formulate the probiotic bacterial strains in formulations which are anhydrous or contain little water, thus making it possible not to resort to preservatives which destroy probiotic bacterial strains by diminishing their viability.

The compositions according to the invention are in addition easy to apply and can be easily formulated, unlike the solutions described in the prior art.

A subject matter of the invention is a cosmetic and/or dermatological composition comprising:
at least one live probiotic bacterial strain,
at least one linear-chain lipodispersible and liposoluble molecule chosen from dicaprylyl carbonate; coco-caprylate, optionally combined with coco-caprate, preferentially a mixture of coco-caprylate and of coco-caprate; undecane and/or tridecane; linoleic acid and/or oleic acid, and/or their linear-chain esters and/or oleyl alcohol; behenyl alcohol and/or its acid and ester derivatives, in particular behenic acid, or docosanoate or behenate esters; and/or their mixtures,
and optionally one or more other linear-chain lipodispersible and liposoluble molecules,
   in which the total content of the linear-chain lipodispersible and liposoluble molecules is at least 40% by weight, with respect to the total weight of the composition.

In particular, in the cosmetic and/or dermatological composition according to the invention, the other linear-chain lipodispersible and liposoluble molecule is chosen from:
alkane molecules of chemical formula CₙH₂ₙ₊₂ in which n denotes a value ranging from 6 to 40,
ester molecules of chemical formula CH₃—CₓH₂ₓ—COO—C_{y}H_{2y}—CH₃ in which each of the x and y indices independently denotes a value ranging from 5 to 19,
acid molecules of chemical formula CH₃—CₓH₂ₓ—C(O)OH in which x denotes a value ranging from 5 to 24, preferably from 5 to 21, such as cerotic acid,
alcohol molecules of chemical formula CH₃—CₓH₂ₓ—OH in which x denotes a value ranging from 5 to 21,
   and any one of their mixtures.

In particular, in the cosmetic and/or dermatological composition according to the invention, the linear-chain lipodispersible and liposoluble molecule chosen from dicaprylyl carbonate; coco-caprylate, optionally combined with coco-caprate, preferentially a mixture of coco-caprylate and of coco-caprate; undecane and/or tridecane; linoleic acid and/or oleic acid, and/or their linear-chain esters and/or oleyl alcohol; behenyl alcohol and/or its acid and ester derivatives, in particular behenic acid, or docosanoate or behenate esters; and/or their mixtures, is present at a content of at least 8%, preferentially at least 10%, more preferentially at least 15%, by weight, with respect to the total weight of the composition.

In particular, in the cosmetic and/or dermatological composition according to the invention, the linear-chain lipodispersible and liposoluble molecule is dicaprylyl carbonate.

In particular, in the cosmetic and/or dermatological composition according to the invention, the linear-chain lipodispersible and liposoluble molecule chosen is coco-caprylate, optionally combined with coco-caprate, preferentially a mixture of coco-caprylate and of coco-caprate.

In particular, in the cosmetic and/or dermatological composition according to the invention, the linear-chain lipodispersible and liposoluble molecule chosen is dicaprylyl carbonate.

In particular, in the cosmetic and/or dermatological composition according to the invention, the linear-chain lipodispersible and liposoluble molecule chosen is undecane and/or tridecane.

In particular, in the cosmetic and/or dermatological composition according to the invention, the linear-chain lipodispersible and liposoluble molecule chosen is linoleic acid and/or oleic acid, and/or isostearyl linoleate and/or oleyl alcohol, preferentially linoleic acid.

In particular, in the cosmetic and/or dermatological composition according to the invention, the linear-chain lipodispersible and liposoluble molecule chosen is behenyl alcohol and/or its acid and ester derivatives, in particular behenic acid, or docosanoate or behenate esters, preferentially behenyl alcohol or docosanol.

In particular, in the cosmetic and/or dermatological composition according to the invention, the linear-chain lipodispersible and liposoluble molecule chosen from dicaprylyl carbonate; coco-caprylate, optionally combined with coco-caprate, preferentially a mixture of coco-caprylate and of coco-caprate; undecane and/or tridecane; linoleic acid and/or oleic acid, and/or their linear-chain esters and/or oleyl alcohol; behenyl alcohol and/or its acid and ester derivatives, in particular behenic acid, or docosanoate or behenate esters; and/or their mixtures, is present at a content of at least 40%, preferentially at least 50%, more preferentially at least 60%, by weight, with respect to the total weight of the composition.

In particular, the cosmetic and/or dermatological composition according to the invention comprises at least two, preferentially three or four, linear-chain lipodispersible and liposoluble molecules chosen from dicaprylyl carbonate; coco-caprylate, optionally combined with coco-caprate, preferentially a mixture of coco-caprylate and of coco-caprate; undecane and/or tridecane; linoleic acid and/or oleic acid, and/or their linear-chain esters and/or oleyl alcohol; behenyl alcohol and/or its acid and ester derivatives, in particular behenic acid, or docosanoate or behenate esters.

In particular, the cosmetic and/or dermatological composition according to the invention additionally comprises a branched-chain liposoluble and lipodispersible molecule chosen from octyldodecanol, propylheptyl caprylate, a mixture of caprylic and capric triglycerides, hydrogenated castor oil, shea butter, coconut oil and/or cocoglycerides.

In particular, in the cosmetic and/or dermatological composition according to the invention, the live probiotic bacterial strain is chosen from Lactobacillus crispatus, Corynebacterium accolens, Sphingomonas glacialis, Streptococcus salivarus and their mixtures.

In particular, in the cosmetic and/or dermatological composition according to the invention, the live bacterial strain is formulated from powder obtained by lyophilization on a maltodextrin support, preferentially ranging from 10% to 80% by weight, with respect to the total weight of powder, preferably from 30% to 70%, more preferentially from 40% to 60%, by weight, with respect to the total weight of powder.

In particular, in the cosmetic and/or dermatological composition according to the invention, the live bacterial strain is at a content of 10³ to 10¹⁵ CFU (colony-forming unit) per gram of composition.

In particular, in the cosmetic and/or dermatological composition according to the invention, the live bacterial strain is at a content of 0.01% to 3% by weight, with respect to the total weight of the composition.

In particular, in the cosmetic and/or dermatological composition according to the invention, the water activity (Aw) in the composition is less than 0.6.

In particular, the cosmetic and/or dermatological composition according to the invention is in the form of an oily suspension or solution, an oily cream or gel, a milk, an emulsion of water-in-oil or multiple or silicone type, a mask, a serum, a lotion, a solid soap, a shampoo in solid form, foaming products in solid form, a dermatological bar, an ointment, a mousse, a patch, an anhydrous product, which is preferably liquid, pasty or solid, for example in the form of make-up powders, rods or sticks.

The present invention relates in addition to the non-therapeutic use of a cosmetic composition according to the invention for preventing and/or reducing unaesthetic and/or uncomfortable manifestations of healthy skin, healthy superficial body growths and/or healthy mucous membranes, in particular of sensitive or sensitized skin, superficial body growths and/or mucous membranes, of fragile or weakened skin, superficial body growths and/or mucous membranes, of dry skin, superficial body growths and/or mucous membranes, and/or of skin, superficial body growths and/or mucous membranes having an atopic tendency.

The present invention additionally relates to the non-therapeutic use of a cosmetic composition according to the invention as moisturizing and/or soothing agent for healthy skin, healthy superficial body growths and/or healthy mucous membranes.

In particular, the non-therapeutic uses according to the invention are carried out by topical application, preferentially to an area of healthy skin, mucous membrane and/or superficial body growth chosen from the legs; the feet; the armpits; the hands; the thighs; the hips; the buttocks; the waist; the crotch; the groin; the stomach; the neckline; the neck; the arms; the torso; the back; the face, including in particular the forehead, the cheeks, the nose, the temples, the T-zone (forehead, nose and chin), the external auditory canal and/or the chin; the scalp; the hair; the nails; and/or the labial mucous membrane, advantageously the armpits, the neckline, the hair, the nails, the labial mucous membrane and/or the face, more advantageously the face.

The present invention additionally relates to the use of at least one linear-chain lipodispersible and liposoluble molecule chosen from dicaprylyl carbonate; coco-caprylate, optionally combined with coco-caprate, preferentially a mixture of coco-caprylate and of coco-caprate; undecane and/or tridecane; linoleic acid and/or oleic acid, and/or their linear-chain esters and/or oleyl alcohol; behenyl alcohol and/or its acid and ester derivatives, in particular behenic acid, or docosanoate or behenate esters; and/or their mixtures, and optionally one or more other linear-chain lipodispersible and liposoluble molecules, for maintaining the viability of a live probiotic bacterial strain, in which the total sum by weight of the linear-chain lipodispersible and liposoluble molecules is at least 40% by weight, with respect to the total weight of a cosmetic or dermatological composition.

The Applicant Company has also discovered that the compositions according to the invention containing at least one live probiotic bacterial strain can thus be used as cosmetic compositions, in particular for improving the appearance and/or comfort of healthy skin, including the healthy scalp and/or mucous membranes and/or healthy superficial body growths, in particular the hair and the nails, and/or for the cosmetic care of healthy skin and/or mucous membranes and/or healthy superficial body growths, including the most delicate skin, such as sensitive, sensitized, fragile, weakened or dry skin, and/or healthy skin having an atopic tendency. The linear-chain liposoluble and lipodispersible molecules selected are emollients which advantageously make it possible in addition to improve the hydration of the skin and/or of the mucous membranes and/or to soften them.

Another subject matter of the invention is a cosmetic care process, characterized in that it comprises the application topically to at least one area of healthy skin and/or mucous membrane and/or healthy superficial body growth of a cosmetic composition according to the invention, for the cosmetic treatment of sensitive healthy skin and/or healthy mucous membranes, sensitized healthy skin and/or healthy mucous membranes, fragile and/or weakened healthy skin and/or healthy mucous membranes, healthy skin and/or healthy dry mucous membranes, healthy skin and/or healthy mucous membranes having an atopic tendency, and/or as soothing agent or moisturizing agent.

The Applicant Company has also discovered that the compositions according to the invention, in particular dermatological compositions, are particularly suitable in the treatment and/or the prevention of infections of the skin, mucous membranes and/or superficial body growths caused by pathogenic microorganisms, in particular mycoses, S. aureus, such as eczema, seborrhoeic or atopic dermatitis, acne, and cutaneous or mucosal inflammation caused by pathogenic bacteria, such as S. aureus, and/or erythema, in particular nappy rash in infants, and/or in the prevention and/or the pharmaceutical treatment, in particular dermatological treatment, of pathologies related to reactive and/or atopic skin.

These compositions according to the invention are thus useful for the dermatological care of atopic and/or reactive skin and/or of infant skin to prevent nappy rash in infants.

A subject matter of the invention is thus a dermatological composition according to the invention, for its use as anti-inflammatory or antibacterial composition and/or in the treatment and/or the prevention of infections of the skin and/or mucous membranes in particular associated with a pathogenic microorganism, such as a yeast or a bacterium, for example mycoses or S. aureus infections, such as eczema, dandruff, seborrhoeic or atopic dermatitis, acne, and cutaneous or mucosal inflammation caused by a pathogenic bacterium, such as S. aureus, and/or erythema, in particular nappy rash in infants, and/or in the prevention and/or the pharmaceutical treatment, in particular dermatological treatment, of pathologies related to reactive and/or atopic skin.

In particular, the invention relates to a dermatological composition according to the invention for its use as anti-inflammatory agent or antibacterial agent and/or in the treatment and/or the prevention of infections of the skin and/or mucous membranes and/or superficial body growths caused by a pathogenic microorganism, such as a pathogenic yeast or bacterium, such as S. aureus, such as mycoses, eczema, seborrhoeic or atopic dermatitis, acne, and cutaneous or mucosal inflammation caused by a pathogenic microorganism, such as a pathogenic yeast or bacterium, such as S. aureus, mycosis and/or erythema, in particular nappy rash in infants, and/or in the prevention and/or the pharmaceutical treatment, in particular dermatological treatment, of pathologies related to reactive and/or atopic skin and/or mucous membranes.

Another subject matter of the invention is a method for the treatment and/or prevention of infections of the skin and/or mucous membranes in particular associated with a pathogenic microorganism, such as a yeast or a bacterium, for example mycoses or S. aureus infections, such as eczema, dandruff, seborrhoeic or atopic dermatitis, acne, and cutaneous or mucosal inflammation caused by a pathogenic bacterium, such as S. aureus, and/or erythema, in particular nappy rash in infants, and/or a method for the prevention and/or pharmaceutical treatment, in particular dermatological treatment, of pathologies related to reactive and/or atopic skin, in a patient having need thereof, comprising the administration to said patient of a pharmaceutically or dermatologically effective amount of a dermatological composition according to the invention.

Another subject matter of the invention is the use of a dermatological composition according to the invention, for the preparation of an anti-inflammatory composition and/or of an antibacterial composition and/or of a medicament for the treatment and/or the prevention of infections of the skin and/or mucous membranes and/or superficial body growths caused by a pathogenic microorganism, such as a pathogenic yeast or bacterium, such as S. aureus, such as mycoses, eczema, seborrhoeic or atopic dermatitis, acne, and cutaneous or mucosal inflammation caused by a pathogenic microorganism, such as a pathogenic yeast or bacterium, such as S. aureus, mycosis and/or erythema, in particular nappy rash in infants, and/or for the prevention and/or the pharmaceutical treatment, in particular dermatological treatment, of pathologies related to reactive and/or atopic skin and/or mucous membranes.

The compositions according to the invention are advantageously intended for topical administration.

Within the meaning of the present invention, the term “cosmetic use” and/or “cosmetic composition” is understood to mean a non-pharmaceutical use and/or composition, that is to say one which is not intended for a therapeutic use and is applied to a “healthy” part of the body, in particular to a “healthy” area of the skin.

Within the meaning of the present invention, the term “healthy skin and/or mucous membrane” is understood to mean an area of human skin and/or mucous membrane to which the agent or the composition according to the invention is applied and referred to as “non-pathological” by a dermatologist, that is to say not exhibiting infection, scar, skin disease or skin condition, such as candidiasis, impetigo, psoriasis, eczema, inflammation, ichthyosis, acne or dermatitis, or wounds or injuries.

Within the meaning of the present invention, the term “skin” is understood to mean the skin of all or part of the body, in particular human body, preferentially chosen from the legs; the feet; the armpits; the hands; the thighs; the hips; the buttocks; the waist; the crotch; the groin; the stomach; the neckline; the neck; the arms; the torso; the back; the face, including the forehead, the cheeks, the nose, the temples, the T-zone (forehead, nose and chin), the external auditory canal and/or the chin; and/or the scalp, more preferentially chosen from the legs, the feet, the armpits, the hands, the thighs, the stomach, the neckline, the neck, the arms, the torso, the back, the face and/or the scalp, advantageously the armpits, the neckline, the face, more advantageously the face.

Within the meaning of the present invention, the term “mucous membrane(s)” is understood to mean the nasal, ocular, vaginal, urogenital and/or buccal mucous membrane, in particular the labial and/or gingival mucous membrane, preferentially the urogenital and/or buccal, preferentially labial, mucous membranes.

According to the present invention, the term “superficial body growths” is understood to mean the hair fiber (the head hair), the eyelashes, the eyebrows, the body hairs, in particular the hairs of the beard and/or of the moustache, and/or the nails, preferably the nails and the head hair.

Within the meaning of the present invention, the term “liposoluble and lipodispersible molecules” is understood to mean molecules which are soluble and dispersible in a liquid oily phase, typically sunflower oil, at ambient temperature (i.e. between 18° C. and 25° C.) or after heating at between 60° C. and 85° C., and at atmospheric pressure.

Within the meaning of the present invention, the term “linear-chain molecule” is understood to mean an alkane, ester, acid, carbonate or alcohol molecule, the carbon-based chain or chains of which do not comprise any branching. The molecule thus comprises one or two linear carbon-based chains, that is to say a saturated or unsaturated carbon-based chain in which each carbon atom is covalently bonded to, at most, two other carbon atoms. It thus does not contain any carbon group branched onto the carbon chain(s).

Within the meaning of the present invention, the term “branched-chain molecule” is understood to mean a molecule, the chain of which comprises at least one branching. The molecule thus comprises at least one branched carbon-based chain, that is to say a saturated or unsaturated carbon-based chain in which at least one of the carbon atoms is covalently bonded to, at least, three other carbon atoms. It can be saturated or unsaturated and contains at least one carbon group branched onto a carbon-based chain.

Within the meaning of the present invention, the term “linear-chain liposoluble and lipodispersible molecules” is understood to mean the following molecules, alone or as a mixture, preferentially at least two of the molecules chosen from the following list:
alkane molecules of chemical formula CₙH₂ₙ₊₂ in which n denotes a value ranging from 6 to 40;
ester molecules of chemical formula CH₃—CₓH₂ₓ—C(O)O—C_{y}H_{2y}—CH₃ in which each of the x and y indices independently denotes a value ranging from 5 to 19;
dicaprylyl carbonate;
acid molecules of chemical formula CH₃—CₓH₂ₓ—C(O)OH in which x denotes a value ranging from 5 to 24, preferably from 5 to 21; and
alcohol molecules of chemical formula CH₃—CₓH₂ₓ—OH in which x denotes a value ranging from 5 to 21.

In particular, the linear-chain liposoluble and lipodispersible molecules can be chosen from dicaprylyl carbonate; coco-caprylate, optionally combined with coco-caprate, preferentially a mixture of coco-caprylate and of coco-caprate; undecane and/or tridecane; linoleic acid and/or oleic acid, and/or their linear-chain esters (e.g.; isostearyl linoleate) and/or oleyl alcohol; behenyl alcohol and/or its acid and ester derivatives, in particular behenic acid, otherwise denoted docosanoic acid, or docosanoate or behenate esters; and/or their mixtures.

Within the meaning of the present invention, the term “selected linear-chain liposoluble and lipodispersible molecules” is understood to mean the following molecules, alone or as a mixture, preferentially at least two of the molecules chosen from the following list: dicaprylyl carbonate; coco-caprylate, optionally combined with coco-caprate, preferentially a mixture of coco-caprylate and of coco-caprate; undecane and/or tridecane; linoleic acid and/or oleic acid, and/or their linear-chain esters (e.g.; isostearyl linoleate) and/or oleyl alcohol; behenyl alcohol and/or its acid and ester derivatives, in particular behenic acid, otherwise denoted docosanoic acid, or docosanoate or behenate esters; and/or their mixtures.

Within the meaning of the present invention, the term “other linear-chain lipodispersible and liposoluble molecules” is understood to mean a linear-chain lipodispersible and liposoluble molecule as defined above but different from the selected linear-chain liposoluble and lipodispersible molecules as also defined above.

The term “derivatives” of an acid molecule according to the present invention is understood to mean the alcohol and ester derivatives.

The term “derivatives” of an alcohol molecule according to the present invention is understood to mean the acid and ester derivatives.

Within the meaning of the present invention, the term “to maintain the viability” is understood to mean to prevent the increase and/or the decrease in the number of colony-forming units (referred to as CFUs) of a live bacterial strain in the composition over time. The viability of a live bacterial strain according to the invention is referred to as “maintained” if the number of CFUs counted after a given time varies at most by 2 log, preferably by one log, more or less, with respect to the number of CFUs initially counted during the formulation of the composition according to the invention.

The maintenance of the viability is preferentially evaluated after a given time ranging from one month to twenty-four months, preferentially at one month, preferentially 3 months, more preferentially 6 months, more preferentially 12 months and/or 16 months and/or 18 months, starting from the formulation of the composition according to the invention.

The maintenance of the viability is preferentially evaluated after storage at a temperature of between 0° C. and 30° C., preferentially between 2° C. and 25° C., preferentially between 2° C. and 6° C. and/or between 10° C. and 30° C. and preferentially at ambient temperature (that is to say between 18° C. and 25° C., in particular approximately 21° C.).

The number of CFUs can be evaluated according to methods conventional in the field, in particular by counting the bacterial strain, in particular by withdrawal of a given amount of composition, placed in an appropriate culture medium, such as the method described in Example 2.

Within the meaning of the present invention, the term “to maintain the stability” of a composition comprising a live bacterial strain is understood to mean preventing the increase or the decrease in the parameters conventionally defined in the field, such as, for example, the organoleptic parameters, such as the color and/or the odor and/or the formulation pharmaceutical parameters, such as the pH, the maintenance of the phases, the absence of precipitation of compounds and/or of growth of another strain of microorganisms.

The live bacterial strain used in the present invention is referred to as probiotic. Within the meaning of the present invention, the term “probiotic or probiotic bacterial strain” is understood to mean one or more strains of bacteria which are living and thus viable, that is to say capable of forming colonies in culture. The bacterial strains referred to as probiotic according to the invention are those of cosmetic and/or dermatological interest and which can be administered topically without risk to humans.

Within the meaning of the present invention, the term “live probiotic bacterial strain” is understood to mean a whole bacterial strain referred to as viable, that is to say a bacterial strain capable of forming colonies in culture.

Within the meaning of the present invention, the term “physiologically acceptable excipient” is understood to mean an excipient suitable for topical application, for cosmetic, pharmaceutical or dermatological use, which is non-toxic and non-irritating to the skin, which does not induce an allergic response and which is not chemically unstable.

The term “topical administration or topically” is understood to mean the direct local application and/or the spraying of the composition onto the surface of the skin, in particular scalp, and/or mucous membranes and/or superficial body growths. Thus, the composition can be applied topically to all or part of the body and/or of the face, advantageously chosen from the legs; the feet; the armpits; the hands; the thighs; the hips; the buttocks; the waist; the crotch; the groin; the stomach; the neckline; the neck; the arms; the torso; the back; the face, including the forehead, the cheeks, the nose, the temples, the T-zone (forehead, nose and chin), the labial mucous membrane, the external auditory canal and/or the chin; the hair; and/or the scalp, more preferentially chosen from the legs, the feet, the armpits, the hands, the thighs, the stomach, the neckline, the neck, the arms, the torso, the back, the face, the hair, the nails and/or the labial mucous membrane and/or the scalp, advantageously the neckline, the face, the hair, the nails and/or the labial mucous membrane, more advantageously the face; the armpits, the neckline, the hair, the nails, the labial mucous membrane, the scalp and/or the face, very advantageously the face.

Because of the complementary emollient properties of the selected liposoluble and lipodispersible molecules according to the present invention, the cosmetic compositions according to the invention are particularly suitable for the cosmetic care and/or treatment of unaesthetic and/or uncomfortable manifestations of any type of skin, in particular skin and/or mucous membranes referred to as normal and/or dry but also sensitive and/or sensitized and/or fragile and/or weakened skin and/or mucous membranes, and/or skin and/or mucous membranes having an atopic tendency, and/or dry skin and/or mucous membranes.

Generally, “sensitive skin and/or mucous membranes” can be defined as healthy skin and/or healthy mucous membranes which, by nature, are only very slightly tolerant of aggressive agents, in particular environmental agents, such as pollutants, climate factors (wind, cold, heat), exposure to UV radiation, emotional factors, in particular stress, and/or chemical agents (heavy metals, detergents, compounds contained in cosmetic treatments, such as fragrances, preservatives, alcohols, pH, AHA or dermatological treatments, such as vitamin A acid) and/or aggressive conditions, in particular perspiration and mechanical attacks, such as hair removal, shaving, rubbing and even water, especially hard water. Sensitive skin is not skin having a pathological character, unlike allergic skin. They can nevertheless react to aggressive agents and/or conditions by unaesthetic and/or uncomfortable cutaneous and/or mucosal manifestations, such as cutaneous and/or mucosal dryness, loss of homogeneity of the complexion of the skin and/or mucus membranes, in particular by the appearance of redness, feelings of tugging, tingling, pins and needles, tightness and/or itching, a rough appearance of the skin and/or mucous membranes and/or a loss of softness to the touch. Thus, the “sensitive skin” character can be assessed by the subject himself/herself with subjective cutaneous sensations or by the dermatologist with objective cutaneous reactions.

The unaesthetic and/or uncomfortable manifestations can be generalized to the whole of the body, but most of the time they can have well-defined locations, such as, for example, the scalp, the face, the skin folds, the buttocks in infants, and the like. It can thus be a matter of areas of sensitive skin and/or mucous membrane.

Likewise, “sensitized skin and/or mucous membranes” are healthy skin and/or healthy mucous membranes rendered momentarily sensitive, thus non-pathological as such.

A “fragile or weakened skin and/or mucous membranes (that is to say, skin or mucous membrane rendered momentarily fragile)” is a healthy skin and/or a healthy mucous membrane, the barrier function of which is weakened. This state may be related to the state of the individual, and/or related to the age, elderly people and infants having fragile skin, for example. This state can also result from chemical or physical attack (abrasion, rubbing actions, cuts).

Likewise, the dermatological compositions according to the invention are particularly suitable for the dermatological care and/or treatment of pathological skin and/or mucous membranes, in particular atopic and/or reactive skin and/or mucous membranes.

“Skin and/or mucous membrane having an atopic tendency” is healthy skin and/or a healthy mucous membrane which is extremely fragile and/or weakened, the permeability of which is increased due to the weakened barrier function and which is genetically predisposed to change under the effect of multiple intrinsic and extrinsic factors towards a pathological state which is atopic dermatitis. Thus, the “skin having an atopic tendency” character can be assessed by the subject himself/herself with subjective cutaneous sensations or by the dermatologist with objective cutaneous reactions.

The uncomfortable and/or unaesthetic manifestations of sensitized, fragile and/or weakened skin and/or mucous membranes are the same as for sensitive skin and/or mucous membrane, without these manifestations and/or skin states coming under the prevention and/or the treatment of a pathology.

“Reactive skin and/or mucous membrane”, otherwise denoted intolerant or irritable or allergic, is skin and/or a mucous membrane, the tolerance threshold of which has decreased and which reacts excessively.

“Atopic skin and/or mucous membrane” is skin and/or a mucous membrane affected by the pathology atopic dermatitis.

The “reactive skin and/or mucous membrane” and “atopic skin and/or mucous membrane” features can be assessed by the dermatologist with objective cutaneous reactions.

In the context of the present invention, the cosmetic and/or dermatological composition comprises:
at least one live probiotic bacterial strain,
at least one selected linear-chain lipodispersible and liposoluble molecule,
and optionally one or more other linear-chain lipodispersible and liposoluble molecules,
   in which the total content of the linear-chain lipodispersible and liposoluble molecules is at least 40% by weight, with respect to the total weight of the composition.

The selected linear-chain liposoluble and lipodispersible molecules according to the present invention are chosen from:
dicaprylyl carbonate,
coco-caprylate, optionally combined with coco-caprate, preferentially a mixture of coco-caprylate and of coco-caprate,
undecane and/or tridecane,
linoleic acid and/or oleic acid, and/or their linear-chain esters (e.g. isostearyl linoleate) and/or oleyl alcohol,
behenyl alcohol and/or its acid and ester derivatives, in particular behenic acid, otherwise denoted docosanoic acid, or docosanoate or behenate esters,
   and/or any one of their mixtures.

This is because, as demonstrated in Example 2, the Applicant Company has observed that these selected linear-chain molecules make it possible to maintain the viability of the probiotic bacterial strain.

In particular, the selected linear-chain liposoluble and lipodispersible molecules according to the invention are:

### Linear-Chain Acids:

dicaprylyl carbonate: otherwise denoted dioctyl carbonate, of formula C17H34O3 and CAS number 1680-31-5; this molecule is a diester of carbonic acid and of caprylyl alcohol. It is conventionally sold by BASF under the name Cetiol® CC or contained in a mixture, such as in the product Cosmedia® Gel CC sold by BASF.
coco-caprylate: of INCI name Coco-Caprylate and otherwise denoted octanoic acid coco alkyl ester (CAS 107525-85-9); this ester having a linear fatty chain is conventionally sold by BASF under the name Cetiol® C5.
coco-caprylate can be combined with coco-caprate, of INCI name Coco-Caprylate/Caprate; this combination, of INCI name Coco-Caprylate/Caprate and CAS number 95912-86, is conventionally sold by BASF under the name Cetiol® C 5C.

These linear-chain acids are known and used in cosmetic and dermatological compositions as emollients.

### Linear-Chain Alkanes:

undecane: it is an alkane of formula C11H24 and of CAS number 1120-21-4
tridecane: this alkane, of formula C13H28 and of CAS number 629-50-5, is conventionally sold by BASF under the name Cetiol® iSan
a mixture of undecane and tridecane is conventionally sold by BASF under the name Cetiol® Ultimate

### a Linear-Chain Fatty Acid or its Derivatives:

linoleic acid: it is a polyunsaturated fatty acid of the omega-6 group. It can in particular be contained in a mixture rich in linoleic acid, such as the Argania spinosa seed oil sold under the name Lipofructyl™ Argan by the Applicant Company. Preferentially, linoleic acid is added in the form contained in a mixture rich in linoleic acid, that is to say containing at least at least 70% thereof, or in pure form.
oleic acid: it is a monounsaturated fatty acid of the omega-9 group. It can in particular be contained in a mixture rich in linoleic acid, that is to say containing at least 50%, preferentially at least 70%, thereof, such as in the form of a rapeseed oil rich in omega-9, such as that sold under the name Cegesoft PS6 by BASF.
the linear-chain esters of linoleic acid or of oleic acid are linear-chain molecules comprising a group having a chain of 20 carbons maximum esterified on the acid function, such as, for example, isostearyl linoleate or decyl oleate.
oleyl alcohol

### A Linear-Chain Fatty Alcohol:

behenyl alcohol: it is otherwise denoted docosanol and of formula C₂₂H₄₆O; it is conventionally sold by BASF under the name Lanette® 22. Its acid and ester derivatives are chosen in particular from behenic acid, otherwise denoted docosanoic acid, its docosanoate or behenate esters and/or their mixtures.

Advantageously, the selected linear-chain liposoluble and lipodispersible molecule(s) is (are) contained in the cosmetic or dermatological composition at a minimum content of 8%, more preferentially at least 10%, preferentially at least 15%, preferentially 40%, more preferentially at least 50% by weight, advantageously at least 60% by weight, more preferentially at 70% by weight, with respect to the total weight of the composition.

The cosmetic or dermatological composition according to the invention advantageously comprises a mixture of at least two, preferentially three or four, of the selected linear-chain liposoluble and lipodispersible molecules, provided that the total content of linear-chain liposoluble and lipodispersible molecules is at least 40%, preferentially at least 50%, more preferentially at least 60%, by weight, with respect to the total weight of the cosmetic or dermatological composition.

According to one embodiment, the selected linear-chain liposoluble and lipodispersible molecules are used as purified molecules or as molecules contained in mixtures containing them, such as extracts. In particular, they are used as purified molecules.

The selected linear-chain liposoluble and lipodispersible molecules according to the invention can be combined with other linear-chain liposoluble and lipodispersible molecules which are not selected, provided that the total content of linear-chain liposoluble and lipodispersible molecules is at least 40%, preferentially at least 50%, more preferentially at least 60%, by weight, with respect to the total weight of the cosmetic or dermatological composition.

These other linear-chain liposoluble and lipodispersible molecules which may be contained in the composition in combination with those selected are preferentially chosen from:
alkane molecules of chemical formula CₙH₂ₙ₊₂ in which n denotes a value ranging from 6 to 40, preferentially one of the paraffins for which n is between 8 and 40, advantageously between 20 and 40 or between 8 and 19, such as, for example, dodecane, tetradecane and/or pentadecane;
ester molecules of chemical formula CH₃—CₓH₂ₓ—COO—C_{y}H_{2y}—CH₃ in which each of the x and y indices independently denotes a value ranging from 3 to 19, such as myristyl myristate C28H56O2, cetearyl palmitate, cetyl palmitate, butyl palmitate or myricyl palmitate;
acid molecules of chemical formula CH₃—CₓH₂ₓ—C(O)OH in which x denotes a value ranging from 5 to 24, preferably from 5 to 21, such as cerotic acid;
alcohol molecules of chemical formula CH₃—CₓH₂ₓ—OH in which x denotes a value ranging from 5 to 21;
   and any one of their mixtures.

According to a preferential embodiment, the present invention relates to a composition intended for topical application, in particular a cosmetic or dermatological composition, comprising at least one live probiotic bacterial strain and at least 40% by weight, with respect to the total composition weight, of one or more selected linear-chain liposoluble and lipodispersible molecules, preferentially at least two selected linear-chain liposoluble and lipodispersible molecules.

Advantageously, the composition according to the invention does not contain paraffin or glycerin.

The cosmetic or dermatological composition according to the invention advantageously comprises a mixture of at least two, preferentially three or four, of the selected linear-chain liposoluble and lipodispersible molecules, the sum of which is at least 40% by weight, preferentially 50% by weight, with respect to the total weight of the cosmetic or dermatological composition.

According to a particular embodiment of the invention, the linear-chain liposoluble and lipodispersible molecules included in the cosmetic or dermatological composition are chosen solely from the selected linear-chain liposoluble and lipodispersible molecules. In other words, the cosmetic or dermatological composition according to the invention is devoid of linear-chain liposoluble and lipodispersible molecule other than those selected.

According to a particular embodiment of the invention, the cosmetic or dermatological composition consists of one or more selected linear-chain liposoluble and lipodispersible molecules and a live probiotic bacterial strain.

The compositions according to the invention contain at least one live probiotic bacterial strain. According to the invention, the term “live probiotic bacterial strain” is understood to mean a strain of bacteria of cosmetic and/or dermatological interest, that is to say which is non-pathogenic and the contribution topically of which can improve the aesthetics and/or the comfort of healthy skin or treat pathologies. It can concern wild-type or genetically modified bacteria.

Preferentially, it concerns the following bacteria:
of the lactic acid bacteria group, in particular Lactobacillus rhamnosus, brevis, crispatus, delbrueckii, fermentum, helveticus, plantarum, iners, gasseri, jensenii, reuteri, brevis and/or acidophilus, preferentially Lactobacillus crispatus;
of the Cocci group, in particular Alloicoccus otitis, Lactococcus lactis, Streptococcus thermophilus, Streptococcus salivarius, Staphylococcus epidermidis, Staphylococcus hominis, Staphylococcus warneri, Staphylococcus caprae and/or Staphylococcus saprophyticus;
of the Micrococcus group, in particular Micrococcus luteus;
of the Bifidobacteria group, in particular Bifidobacterium longum and/or Bifidobacterium infantis;
of the Corynebacterium group, in particular Corynebacterium accolens, Corynebacterium tuberculostearicum and/or Corynebacterium amycolatum;
of the Sphingomonas group, in particular Sphingomonas glacialis;
of the Bacillus group, in particular Bacillus cereus (subtilis) and/or Bacillus lichenmformis; and
of the Cutibacterium group, in particular Cutibacterium acnes.

More preferentially, the following bacteria are concerned:
of the lactic acid bacteria group, in particular Lactobacillus rhamnosus, brevis, crispatus, delbrueckii, fermentum, helveticus, plantarum, iners, gasseri, jensenii, reuteri, brevis and/or acidophilus, preferentially Lactobacillus crispatus;
of the Cocci group, in particular Alloicoccus otitis, Lactococcus lactis, Streptococcus thermophilus, Streptococcus salivarius, Staphylococcus epidermidis and/or Staphylococcus hominis;
of the Bifidobacteria group, in particular Bifidobacterium longum and/or Bifidobacterium infantis;
of the Corynebacterium group, in particular Corynebacterium accolens;
of the Sphingomonas group, in particular Sphingomonas glacialis;
of the Bacillus group, in particular Bacillus cereus (subtilis) and/or Bacillus licheniformis;
of the Cutibacterium group, in particular Cutibacterium acnes.

Preferentially, the live probiotic bacterial strain is chosen from Lactobacillus crispatus, Corynebacterium accolens, Sphingomonas glacialis, Streptococcus salivarus and their mixtures.

Particularly advantageously, the live probiotic bacterial strain is one of the following strains deposited according to the Budapest Treaty at the Institut Pasteur (25 rue du Docteur Roux, F-75724 Paris Cedex 15):
the Lactobacillus crispatus strain deposited under the number CNCM I-5579,
the Sphingomonas glacialis strain deposited under the number CNCM I-5829.

The live probiotic bacterial strain can be formulated in the powder state, that is to say in dry form, or in the form of suspensions.

In general, the live probiotic bacterial strain can be isolated before it is put into a composition, that is to say not mixed with one or more compounds of its medium of origin. Alternatively, the live probiotic bacterial strain can be combined with compounds of its medium of origin, in particular its culture medium.

According to a preferential embodiment, the live probiotic bacterial strain can be combined with a bacterial strain in inactivated form, in particular in dead form and/or in the form of its lysate, and/or in the form of one or more of its fractions and/or in the form of one or more of its metabolites, in particular its secretome. It can thus concern the same bacterial strain in inactivated form or another bacterial strain in inactivated form. Preferentially, it will concern the same bacterial strain as the live probiotic bacterial strain.

According to one embodiment, the compositions according to the invention can contain several live probiotic bacterial strains or a live probiotic bacterial strain combined with another bacterial strain in inactivated form, in particular in dead form and/or in the form of its lysate, and/or in the form of one or more of its fractions and/or in the form of one or more of its metabolites, in particular its secretome.

Particularly advantageously, the live probiotic bacterial strain is contained in the cosmetic or dermatological composition at a concentration of 0.001% to 3% (w/w) by weight, more preferentially at a concentration of 0.01% to 0.3% (w/w) of approximately 0.025% (w/w) by weight, with respect to the total weight of the composition.

Advantageously, the live probiotic bacterial strain is contained in the cosmetic or dermatological composition at a concentration per gram of composition ranging from 10³ colony-forming units (CFU) to 10¹⁵ CFU/g, preferentially ranging from 10⁴ to 10¹⁰ CFU/g, more preferentially ranging from 10⁵ to 10⁹ CFU/g, more preferentially ranging from 10⁵ to 10⁸ CFU/g, more preferentially of approximately 10⁵ CFU/g.

Advantageously, the live probiotic bacterial strain is contained in the cosmetic composition at a concentration per gram of composition ranging from 10³ to 10⁸ CFU/g, more preferentially ranging from 10⁴ to 10⁸ CFU/g, more preferentially ranging from 10⁵ to 10⁸ CFU/g, more preferentially of approximately 10⁵ CFU/g.

Advantageously, the live probiotic bacterial strain is contained in the dermatological composition at a concentration per gram of composition ranging from 10⁹ colony-forming units (CFU) to 10¹⁵ CFU/g, preferentially ranging from 10⁹ to 10¹³ CFU/g.

The production of the live probiotic bacterial strain can be carried out according to any method conventionally known to a person skilled in the art. Advantageously, it will be carried out according to the protocol as described, for example, in Example 1.

According to an advantageous embodiment, the cosmetic or dermatological composition according to the invention has a water activity of less than 0.6, more preferentially of less than 0.5, more preferentially of less than 0.4.

The water activity (AW) indicates the availability of so-called “free” water of a matrix. It is thus not the exact water content of a medium but only the water capable of participating in chemical, biochemical or microbiological reactions. It is defined with respect to a reference state which is pure water, thus without any solute. The value of the AW varies between 0 and 1. It is equal to 0 when the product is dry and all the water is bound to the matrix (without reactive quality). It is equal to 1 in the case of pure water.

The water activity is measured in a way conventionally accepted by a person skilled in the art with an apparatus for measuring the water activity, such as, for example, the Aqualab apparatuses sold by Metergroup.

According to a more preferential embodiment, the composition according to the invention contains a maximum of 5% by weight of added water, with respect to the total weight of the composition, preferentially less than 3% and more preferentially does not contain added water. According to a particular embodiment, the composition according to the invention is a non-aqueous composition.

According to a particular embodiment of the invention, the live probiotic bacterial strain is included in an oily phase (or non-aqueous phase). Alternatively, the live probiotic bacterial strain according to the invention can be used dispersed and/or diluted in a solvent. Preferentially, this solvent contains less than 20% (v/v) by volume of water, more preferentially less than 5% (v/v) by volume of water, more preferentially, the solvent does not contain water.

According to a preferential embodiment, this solvent contains less than 20% (w/w) by weight of water, more preferentially less than 5% (w/w) by weight of water, more preferentially, the solvent does not contain water. Very preferentially, this solvent is one of the selected liposoluble and lipodispersible molecules according to the invention.

The live probiotic bacterial strain according to the invention is added to the composition according to the invention preferentially in dry form, that is to say in powder form, advantageously in maltodextrin, preferentially in a strain content of 10% to 80% (w/w) by weight of the strain, preferentially of 30% to 70% (w/w), more advantageously of 40% to 60% (w/w) by weight of the strain, with respect to the total weight of powder.

According to a particular embodiment, the cosmetic or dermatological composition according to the present invention in its implementation can comprise:
at least one live probiotic bacterial strain chosen from Lactobacillus crispatus, Corynebacterium accolens, Sphingomonas glacialis, Streptococcus salivarus and their mixtures, preferentially the Lactobacillus crispatus strain deposited under the number CNCM I-5579 and Sphingomonas glacialis, preferentially the strain deposited under the number CNCM I-5829;
said live probiotic bacterial strain being preferentially:
   at a content ranging from 10⁴ to 10¹⁰ CFU/g, more preferentially ranging from 10⁵ to 10⁹ CFU/g, more preferentially ranging from 10⁵ to 10⁸ CFU/g of the composition,
   added to the composition in the form of a freeze-dried powder on a maltodextrin support at a strain content ranging from 40% to 60% (w/w) by weight of the strain, with respect to the total weight of maltodextrin powder,
at least one linear-chain liposoluble and lipodispersible molecule chosen from dicaprylyl carbonate; coco-caprylate, optionally combined with coco-caprate, preferentially a mixture of coco-caprylate and of coco-caprate; undecane and/or tridecane; linoleic acid and/or oleic acid, and/or their linear-chain esters (e.g.; isostearyl linoleate) and/or oleyl alcohol; behenyl alcohol and/or its acid and ester derivatives, in particular behenic acid, otherwise denoted docosanoic acid, or docosanoate or behenate esters; and any one of their mixtures;
at least one other linear-chain liposoluble and lipodispersible molecule; and
a total content of linear-chain liposoluble and lipodispersible molecule of at least 40%, preferentially 50%, by weight, with respect to the total weight of the composition.

The cosmetic or dermatological composition according to the present invention in its preferential implementation comprises:
at least one live probiotic bacterial strain chosen from Lactobacillus crispatus, Corynebacterium accolens, Sphingomonas glacialis, Streptococcus salivarus and their mixtures, preferentially the Lactobacillus crispatus strain deposited under the number CNCM I-5579 and Sphingomonas glacialis, preferentially the strain deposited under the number CNCM I-5829;
said live probiotic bacterial strain being preferentially:
   at a content ranging from 10⁴ to 10¹⁰ CFU/g, more preferentially ranging from 10⁵ to 10⁹ CFU/g, more preferentially ranging from 10⁵ to 10⁸ CFU/g of the composition,
   added to the composition in the form of a freeze-dried powder on a maltodextrin support at a strain content ranging from 40% to 60% (w/w) by weight of the strain, with respect to the total weight of maltodextrin powder,
at least one, preferably at least two, linear-chain liposoluble and lipodispersible molecule(s) chosen from dicaprylyl carbonate; coco-caprylate, optionally combined with coco-caprate, preferentially a mixture of coco-caprylate and of coco-caprate; undecane and/or tridecane; linoleic acid and/or oleic acid, and/or their linear-chain esters (e.g.; isostearyl linoleate) and/or oleyl alcohol; behenyl alcohol and/or its acid and ester derivatives, in particular behenic acid, otherwise denoted docosanoic acid, or docosanoate or behenate esters; and any one of their mixtures;
a total content of selected linear-chain liposoluble and lipodispersible molecule of at least 40%, preferentially 50%, by weight, with respect to the total weight of the composition.

The cosmetic and dermatological compositions are produced according to conventional formulation methods.

Preferentially, the live probiotic bacterial strain is preferentially directly dispersed in the linear- and/or branched-chain liposoluble and lipodispersible molecules, preferentially the linear-chain ones, more preferentially the selected linear-chain ones, that is to say those chosen from dicaprylyl carbonate; coco-caprylate, optionally combined with coco-caprate, preferentially a mixture of coco-caprylate and of coco-caprate; undecane and/or tridecane; linoleic acid and/or oleic acid, and/or their linear-chain esters (e.g.; isostearyl linoleate) and/or oleyl alcohol; behenyl alcohol and/or its acid and ester derivatives, in particular behenic acid, otherwise denoted docosanoic acid, or docosanoate or behenate esters; and any one of their mixtures.

Preferentially, the composition according to the invention is produced at a maximum temperature of 70° C. in the context of “hot” manufacturing or at ambient temperature, that is to say between 18° C. and 25° C., in particular approximately 21° C., in the context of “cold” manufacturing.

Advantageously, the manufacturing temperature does not exceed 70° C. for a maximum duration of 3 hours. It will be adjusted according to the resistance of the live bacterial strain concerned.

In a preferential embodiment, the composition according to the invention will be produced at ambient temperature, that is to say between 18° C. and 25° C., in particular 21° C., in the context of “cold” manufacturing.

The cosmetic and dermatological composition according to the invention is a composition for topical administration.

In an advantageous embodiment, the composition according to the invention additionally comprises a physiologically acceptable excipient, in particular a cosmetically, pharmaceutically or dermatologically acceptable excipient.

In the context of the invention, the live probiotic bacterial strain is used as active agent in the composition, in particular as cosmetic or dermatological active ingredient, for preventing and/or reducing unaesthetic and/or uncomfortable manifestations of healthy skin and/or healthy mucous membranes referred to as normal, sensitive and/or sensitized and/or fragile and/or weakened and/or dry and/or having an atopic tendency.

The cosmetic or dermatological composition according to the invention can also be used in the form of a cosmetic or dermatological agent or of a premix intended to be incorporated in another cosmetic or dermatological composition, and additionally comprising an appropriate cosmetic or pharmaceutical excipient, in particular just before its immediate application.

In this case, according to a particularly advantageous embodiment for this mode of administration, the live probiotic bacterial strain is then contained in the composition according to the invention at a more concentrated dose, in particular at a concentration ranging from 0.001% to 3% by weight, more preferentially at a concentration of 0.01% to 0.3% by weight, preferentially of 0.1% to 0.25%, more preferentially at a concentration of approximately 0.20% by weight, with respect to the total weight of the composition.

Preferentially, the composition according to the invention is a ready-for-use cosmetic or dermatological composition, that is to say a composition which can be used directly by topical application.

The composition according to the invention does not comprise an agent at a biocidal dose and/or preservative which can detrimentally affect the viability of the live probiotic strain.

The compositions according to the invention can contain any suitable solvent, excipient or vehicle, and also other active ingredients, provided that it does not detrimentally affect the stability of the composition, in particular the viability of the live probiotic bacterial strain.

Advantageously, the composition according to the invention can also contain non-linear-chain liposoluble and lipodispersible molecules, in particular branched-chain fatty substances, provided that the linear-chain liposoluble and lipodispersible molecule(s), in particular those selected, are present in the cosmetic composition at a minimum content of 40%, preferentially of at least 50% by weight, more advantageously of at least 60% by weight, more preferentially of at least 70% by weight, with respect to the total weight of the composition.

The term “branched-chain fatty substance” is understood generally to mean liquid, pasty or solid oily compounds, in particular waxes, with a branched chain, in particular a vegetable oil, and also saturated fatty acids with one or more hydrocarbon-based branchings on the carbon-based chain (conventionally denoted BCFA (branched-chain fatty acid)). These branched-chain fatty substances are commonly used in a great many cosmetic and dermatological compositions as texturing agent, emollient, structuring agent or rheology-modifying agent.

The branched-chain fatty substance(s) is (are) advantageously those chosen from oils of fatty substance type with a branched chain, such as:
octyldodecanol, otherwise denoted Eutanol® G;
propylheptyl caprylate, sold in particular by BASF under the name Cetiol® Sensoft;
a mixture of caprylic and capric triglycerides known under the CAS number 73398-61-5, such as that sold by BASF under the name Myritol® 318;
hydrogenated castor oil;
shea butter or other fatty acids, such as the mixture sold by BASF under the name Cetiol® SB 45 comprising oleic, stearic, linolenic, palmitic, linoleic and arachidonic acids;
coconut oil and/or cocoglycerides, in particular those sold by BASF under the name Myritol® 331;
squalane or squalane derivatives, such as hemisqualane.

Preferentially, the composition according to the invention contains a maximum amount of 40%, preferentially 30%, by weight of branched-chain fatty substances, in particular those listed above, more preferentially less than 20% by weight, with respect to the total weight of the composition. This is because, according to this preferential embodiment, the composition exhibits an improved viability at ambient temperature, as demonstrated in Example 6.

According to a particular embodiment of the invention, the cosmetic or dermatological composition is devoid of non-linear-chain liposoluble and lipodispersible molecules and thus of branched-chain fatty substances.

Other liposoluble ingredients can be contained in the compositions according to the invention, such as, for example:
vitamins or antioxidants, in particular vitamin E, otherwise denoted tocopherol, to prevent oxidation of the fatty substances;
feel agents, such as microcrystalline celluloses;
texturing agents, such as fatty-phase thickeners and/or gelling agents, such as, for example, stearalkonium hectorite and propylene carbonate, in particular sold in the form of the Cosmedia® Gel CC mixture sold by BASF in combination with dicaprylyl carbonate;
other emollients.

The excipient(s) can thus be chosen from surface-active agents and/or emulsifiers, buffers, chelating agents, denaturing agents, opacifying agents, pH adjusters, reducing agents, stabilizing agents, thickeners, gelling agents, film-forming polymers, fillers, matifying agents, shine agents, pigments, dyes, fragrances and their mixtures. The CTFA (Cosmetic Ingredient Handbook, Edition 2016) describes various cosmetic excipients suitable for use in the present invention.

Advantageously, the excipient(s) is (are) chosen from the group comprising polyglycerols, esters, cellulose polymers and derivatives, lanolin derivatives, phospholipids, lactoferrins, lactoperoxidases, sucrose-based stabilizers, vitamin E and its derivatives, xanthan gums, natural and synthetic waxes, vegetable oils, triglycerides, non-saponifiable substances, phytosterols, silicones, protein hydrolysates, betaines, amine oxides, plant extracts, saccharose esters, titanium dioxides, glycines, and more preferably from the group consisting of steareth-2, steareth-21, glycol-15 stearyl ether, cetearyl alcohol, butylene glycol, caprylyl glycol, natural tocopherols, glycerin, sodium dihydroxycetyl phosphate, isopropyl hydroxycetyl ether, glycol stearate, triisononanoin, octyl cocoate, polyacrylamide, isoparaffin, laureth-7, a carbomer, propylene glycol, hexylene glycol, glycerol, bisabolol, a dimethicone, sodium hydroxide, PEG-30 dipolyhydroxystearate, capric/caprylic triglycerides, cetearyl octanoate, dibutyl adipate, grape seed oil, jojoba oil, magnesium sulfate, EDTA, a cyclomethicone, xanthan gum, citric acid, sodium lauryl sulfate, mineral oils and waxes, isostearyl isostearate, propylene glycol dipelargonate, propylene glycol isostearate, PEG 8, beeswax, hydrogenated palm kernel oil glycerides, lanolin oil, sesame oil, cetyl lactate, lanolin alcohol, castor oil, titanium dioxide, lactose, saccharose and their mixtures.

According to a particularly advantageous embodiment, the composition according to the invention also comprises biodegradable polymers chosen from:
alginic acid, its salts and its esterified derivatives which are topically acceptable, advantageously chosen from ammonium alginate, sodium alginate, calcium alginate, magnesium alginate, sodium alginate sulfate and potassium alginate, glyceryl alginate or propylene glycol alginate, or their mixtures, advantageously sodium alginate and/or propylene glycol alginate;
hyaluronic acid, its salts and its esterified derivatives which are topically acceptable, advantageously chosen from hydrolyzed calcium hyaluronate, hydrolyzed sodium hyaluronate, potassium hyaluronate, sodium hyaluronate or sodium hyaluronate sulfate, preferentially sodium hyaluronate;
scleroglucan;
   and/or any one of their mixtures.

According to a preferential embodiment of the present invention, it has been discovered that such polymers further increase the viability of the live probiotic bacterial strain in the composition according to the invention, in particular when they are present at a content of between 0.1% and 10%, more preferentially between 1% and 5%, by weight, with respect to the total weight of the cosmetic or dermatological composition.

The cosmetic or dermatological composition according to the invention can be chosen from an oily suspension or solution, an oily cream or gel, a milk, an emulsion of water-in-oil or multiple or silicone type, a mask, a serum, a lotion, a solid soap, a shampoo in solid form, foaming products in solid form, a dermatological bar, an ointment, a mousse, a patch, an anhydrous product, which is preferably liquid, pasty or solid, for example in the form of make-up powders, rods or sticks.

Advantageously, the cosmetic or dermatological composition according to the invention is not in the form of a water-in-oil emulsion.

The cosmetic or dermatological composition can in addition comprise a cosmetically or dermatologically acceptable excipient chosen from surface-active agents, buffers, swelling agents, chelating agents, denaturing agents, opacifying agents, pH adjusters, reducing agents, stabilizing agents, emulsifiers, thickeners, gelling agents, film-forming polymers, solvents, fillers, odor absorbers, matifying agents, conditioning agents, texturing agents, shine agents, pigments, dyes, fragrances, chemical or inorganic sunscreens, trace elements and essential oils. These combinations are also covered by the present invention.

The cosmetic or dermatological composition can contain other liposoluble and lipodispersible active ingredients.

The cosmetic or dermatological composition can in addition comprise other ingredients which are active with regard to the treatment of normal skin and/or mucous membranes, sensitive, sensitized, reactive, fragile and/or weakened skin and/or mucous membranes and/or having an effect on the increase and/or the protection and/or the maintenance of the beneficial commensal flora on the skin and/or the mucus membranes, inducing a complementary or synergistic effect with the probiotic bacterial strain according to the invention, for example chosen from:
a combination of sodium hyaluronate, pullulan and sodium alginate in particular sold in a formulation containing serine, trehalose, urea and glycerin under the name PatcH2O™ or a combination of hyaluronic acid, pullulan and propylene glycol alginate with lactic acid, succinic acid and/or sucrose;
other cosmetic ingredients intended for the care of sensitive skin, such as a plant extract of Cestrum latifolium, such as described in Application WO2009/112590, for example sold under the name Symbiocell™ by the Applicant, a butter extracted from the fruit of the tree Irvingia gabonensis sold under the name Irwinol™ by the Applicant, an extract of Eperua falcata root sold under the name Eperuline™, a peptide N-acetyl-L-tyrosyl-L-prolyl-L-phenylalanyl-L-phenylalaninamide (INCI: Acetyl Tetrapeptide-15) sold under the name Skinasensyl™ by the Applicant.

The cosmetic composition will also be able to contain one or more ingredients which are active with regard to the cutaneous and/or mucosal microbial flora and/or active with regard to the barrier function of the skin, in particular moisturizing and/or soothing active ingredients, among them an oligosaccharide obtained by enzymatic synthesis sold by Solabia under the name BioEcolia™ or a complex of α-glucooligosaccharides sold by the same company under the name Ecoskin™, an extract of Alisma plantago-aquatica, an extract of Argania spinosa (Lipofructyl™ Argan), a mixture of ceramides (Sphingoceryl™ VEG), purifying extracts of boldo (Betapur™), products based on inulin or on fructooligosaccharides, extracts of bifidobacteria or also an extract of Orthosiphon stamineus to combat oily skin (MAT-XS™ Bright), a natural extract of honey sold by the Applicant Company under the name Melhydran™ for its moisturizing property, a flax extract sold under the name Oligolin™ by the Applicant Company, an extract of the seeds of Cassia angustifolia and sold by the Applicant Company under the name Hyalurosmooth®, a yeast extract modified by biotechnology and sold by the Applicant Company under the name Relipidium™, Hydagen GG an extract of Pueraria lobata root sold under the name Inhipase™ by the Applicant Company, a β-glucan derivative resulting from baker's yeast sold by Mibelle under the name CM-Glucan Forte™ and/or an extract of Mirabilis jalapa sold under the name Pacifeel™ by Sederma, inulin, N-methylglycine as cutaneous prebiotic sold in particular under the name Scalposine™ by the Applicant Company, an agent for repairing the barrier function extracted from phytosterols of Brassica campestris seed oil sold by the Applicant Company under the name Phytosoothe™, a moisturizing and soothing agent containing β-glucans and sold under the name Hydrasensyl Glucan™ by the Applicant Company, a moisturizing agent comprising hyaluronic acid and konjac polyssacharides sold under the name Ultra Filling Spheres™, an antioxidant, anti-inflammatory, anti-redness agent containing an extract of Inonotus obliquus sold by the Applicant Company under the name Inolixirm soothing, an agent which protects against the effects of pollution, in particular an extract of Moringa oleifera seeds sold by the Applicant Company under the name Purisoft™, an extract of Cassia angustsfolia sold by the Applicant Company under the name Hyalurosmooth®, a soybean extract fermented by a lactobacillus sold by the Applicant Company under the name Phytofum™ Biotic. According to a particularly advantageous embodiment, the active ingredient which is contained in the composition is of oily and/or lipodispersible nature, such as an oily extract of Cyperus esculentus, sold by the Applicant Company under the name Lipofructyl® Cyperus, an extract of Argania spinosa fruit sold by the Applicant Company under the name Argassential™, an extract of the fruit of Schisandra chinensis, dehydrated oily spheres of hyaluronic acid sold under the name Hyaluronic Filling Spheres.

Particularly advantageously, the cosmetic or dermatological composition according to the invention also comprises an extract of the fermentation medium of the probiotic bacterial strain.

Another subject matter of the present invention is the use of at least one linear-chain liposoluble and lipodispersible molecule chosen from dicaprylyl carbonate; coco-caprylate, optionally combined with coco-caprate, preferentially a mixture of coco-caprylate and of coco-caprate; undecane and/or tridecane; linoleic acid and/or oleic acid, and/or their linear-chain esters (for example; isostearyl linoleate) and/or oleyl alcohol; behenyl alcohol and/or its acid and ester derivatives, in particular behenic acid, otherwise denoted docosanoic acid, or docosanoate or behenate esters; or any one of their mixtures, for maintaining the viability of a live probiotic bacterial strain in a cosmetic or dermatological composition according to the invention, in which said molecule or its mixture is present at a content of at least 40% by weight, with respect to the total weight of the composition.

This use is particularly interesting and advantageous in the presence of a cosmetic or dermatological excipient, especially when the composition contains one or more branched-chain fatty substances, in particular those defined above, especially when these have a content of greater than 10%, preferentially 20%, by weight, with respect to the total weight of the composition, and preferentially between 20% and 40% by weight, with respect to the total weight of the composition.

The present invention also relates to the non-therapeutic cosmetic use topically of a cosmetic composition according to the invention, for the cosmetic treatment of normal healthy skin and/or healthy superficial body growths and/or healthy mucous membranes, sensitive healthy skin and/or healthy superficial body growths and/or healthy mucous membranes, sensitized healthy skin and/or healthy superficial body growths and/or healthy mucous membranes, fragile healthy skin and/or healthy superficial body growths and/or healthy mucous membranes, weakened healthy skin and/or healthy superficial body growths and/or healthy mucous membranes, healthy dry skin and/or superficial body growths and/or mucous membranes and/or healthy skin and/or healthy mucous membranes having an atopic tendency.

The invention additionally relates to a cosmetic care process, characterized in that it comprises the application topically to at least one area of healthy skin and/or healthy mucous membrane of a cosmetic composition according to the invention, for the cosmetic treatment of normal healthy skin and/or healthy superficial body growths and/or healthy mucous membranes, sensitive healthy skin and/or healthy superficial body growths and/or healthy mucous membranes, sensitized healthy skin and/or healthy superficial body growths and/or healthy mucous membranes, fragile and/or weakened healthy skin and/or healthy superficial body growths and/or healthy mucous membranes, dry healthy skin and/or healthy superficial body growths and/or healthy mucous membranes, and healthy skin and/or healthy mucous membranes having an atopic tendency.

In an advantageous embodiment of the invention, the cosmetic care process comprises the topical application of the cosmetic composition according to the invention to all or part of the body and/or of the face, advantageously chosen from the legs; the feet; the armpits; the hands; the thighs; the hips; the buttocks; the waist; the crotch; the groin; the stomach; the neckline; the neck; the arms; the torso; the back; the face, including the forehead, the cheeks, the nose, the temples, the T-zone (forehead, nose and chin), the labial mucous membrane, the external auditory canal and/or the chin; the hair; and/or the scalp, more preferentially chosen from the legs, the feet, the armpits, the hands, the thighs, the stomach, the neckline, the neck, the arms, the torso, the back, the face, the hair, the nails and/or the labial mucous membrane and/or the scalp, advantageously the neckline, the face, the hair, the nails and/or the labial mucous membrane, more advantageously the face; the armpits, the neckline, the hair, the nails, the labial mucous membrane, the scalp and/or the face, very advantageously the face.

In one embodiment, the amount of live probiotic bacterial strain contained in the cosmetic composition does not provide a therapeutic effect.

Advantageously, another subject matter of the invention is a cosmetic (non-therapeutic) treatment method for preventing and/or reducing unaesthetic and/or uncomfortable manifestations of healthy skin and/or healthy superficial body growths and/or healthy mucous membranes which are sensitive and/or sensitized and/or fragile and/or weakened and/or dry and/or having an atopic tendency of an individual who has need of it/who desires it comprising the stages:
a) the identification on the individual of an area of healthy skin and/or mucous membrane and/or superficial body growth, the unaesthetic and/or uncomfortable manifestations of which it is desired to prevent and/or reduce and/or the sensitivity and/or the fragility and/or the dryness and/or the atopic tendency of which it is desired to prevent and/or reduce, and
b) the topical application to this area of healthy skin and/or healthy superficial body growth and/or healthy mucous membrane of a cosmetic composition according to the invention in an amount effective for preventing and/or reducing unaesthetic and/or uncomfortable manifestations on this area of skin, superficial body growth and/or mucous membrane and/or in an amount effective for preventing and/or reducing its sensitivity and/or its fragility and/or its dryness and/or its atopic tendency.

The invention additionally relates to a dermatological composition as defined above, optionally with a cosmetically and/or dermatologically acceptable excipient.

The cosmetically and/or dermatologically acceptable excipient(s) can be chosen from surface-active agents and/or emulsifiers, buffers, chelating agents, denaturing agents, opacifying agents, pH adjusters, reducing agents, stabilizing agents, thickeners, gelling agents, film-forming polymers, fillers, matifying agents, shine agents, pigments, dyes, fragrances and their mixtures. The CTFA (Cosmetic Ingredient Handbook, Edition 2016) describes various cosmetic excipients suitable for use in the present invention. A person skilled in the art knows how to adapt the formulation of the composition according to the invention by using his/her general knowledge.

The invention additionally relates to a dermatological composition according to the invention, as defined above, for its use as anti-inflammatory agent or antibacterial agent and/or in the treatment and/or the prevention of infections of the skin and/or mucous membranes and/or superficial body growths caused by a pathogenic microorganism, such as a pathogenic yeast or bacterium, such as S. aureus, such as mycoses, eczema, seborrhoeic or atopic dermatitis, acne, and cutaneous or mucosal inflammation caused by a pathogenic microorganism, such as a pathogenic yeast or bacterium, such as S. aureus, mycosis and/or erythema, in particular nappy rash in infants, and/or in the prevention and/or the pharmaceutical treatment, in particular dermatological treatment, of pathologies related to reactive and/or atopic skin and/or mucous membranes.

Another subject matter of the invention is a method for the treatment and/or prevention of infections of the skin and/or mucous membranes and/or superficial body growths induced by a pathogenic microorganism, such as a pathogenic yeast or bacterium, such as S. aureus, such as mycoses, eczema, seborrhoeic or atopic dermatitis, acne, and cutaneous or mucosal inflammation caused by a pathogenic microorganism, such as a pathogenic yeast or bacterium, such as S. aureus, mycosis and/or erythema, in particular nappy rash in infants, and/or a method for the prevention and/or pharmaceutical treatment, in particular dermatological treatment, of pathologies related to reactive and/or atopic skin, in a patient having need thereof, comprising the administration to said patient of a pharmaceutically or dermatologically effective amount of a dermatological composition according to the invention.

Another subject matter of the invention is the use of a dermatological composition according to the invention, for the preparation of an anti-inflammatory composition and/or of an antibacterial composition and/or of a medicament for the treatment and/or the prevention of infections of the skin and/or mucous membranes and/or superficial body growths caused by a pathogenic microorganism, such as a pathogenic yeast or bacterium, such as S. aureus, such as mycoses, eczema, seborrhoeic or atopic dermatitis, acne, and cutaneous or mucosal inflammation caused by a pathogenic microorganism, such as a pathogenic yeast or bacterium, such as S. aureus, mycosis and/or erythema, in particular nappy rash in infants, and/or for the prevention and/or the pharmaceutical treatment, in particular dermatological treatment, of pathologies related to reactive and/or atopic skin and/or mucous membranes.

In an advantageous embodiment, the live probiotic bacterial strain is contained in the dermatological composition at a concentration per gram of composition ranging from 10⁹ colony-forming units (CFU) to 10¹⁵ CFU/g, preferentially ranging from 10⁹ to 10¹³ CFU/g.

The invention will now be illustrated with the help of the examples below. Other aims, features and advantages of the invention will become clearly apparent to a person skilled in the art after reading the explanatory description which refers to examples. These are presented by way of indication and do not in any way limit the invention.

The examples form an integral part of the present invention and any feature appearing novel with respect to any state of the prior art from the description taken in its entirety, including the examples, forms an integral part of the invention in its function and in its general nature.

Thus, each example has a general scope.

Moreover, in the examples and unless otherwise indicated, the temperature is expressed in degrees Celsius and the pressure is atmospheric pressure.

### EXAMPLES

### Example 1: Preparation of a Composition According to the Invention

Isolated Lactobacillus crispatus bacteria (CNCM I-5579) were inoculated in a culture medium of MRS type (Man, Rogosa, Sharpe agar) and incubated at 37° C. anaerobically. The pH was maintained at a value of pH=6 and the medium was incubated for 24h. At the end of incubation, the whole of the medium, including the bacteria, was centrifuged, the cell concentrate was recovered and freeze-dried in the presence of maltodextrin, for a final amount of maltodextrin of 50% (w/w) by weight with respect to the total weight of the mixture.

The powder obtained, composed of the mixture having 50% by weight of the L. crispatus strain and 50% by weight of maltodextrin, is dispersed at 0.425% (w/w) in coco-caprylate (Cetiol® C5 sold by BASF) in an amount sufficient to reach 100% (w/w) of the composition.

### Example 2: Monitoring of the Viability of the Live Probiotic Bacteria in a Composition According to the Invention

### Protocol:

The composition according to the invention was obtained according to Example 1.

The composition tested was stored at 4° C. (T4° C.) and at ambient temperature (Tambient, that is to say between 18° C. and 25° C., in particular approximately 21° C.). At the start of the study (T0) and at regular time intervals (T1month, T2months, T3months, T6months, T12months and T16months), the composition is withdrawn and spread over an MRS (Man Sharpe Rugosa) agar. The agars are incubated in anaerobic jars at 37° C. for 3 days and then the colonies are counted. The results are expressed in CFU (colony-forming units) per gram of composition in Table 1 ([Table 1]).

### Results:

TABLE 1

| Composition | | | | | | | |
|---|---|---|---|---|---|---|---|
| according to | | | | | | | |
| the invention | | | | | | | |
| (CFU/g) | T0 | T1 month | T2 months | T3 months | T6 months | T12 months | T16 months |
| T at 4° C. | 2.25 × 10⁷ | 2.1 × 10⁷ | 1.9 × 10⁷ | 2.3 × 10⁷ | 5.6 × 10⁷ | 1.1 × 10⁷ | 2.2 × 10⁷ |
| Tambient | 2.25 × 10⁷ | 1.5 × 10⁷ | 2.8 × 10⁷ | 1.8 × 10⁷ | 1.7 × 10⁷ | 2.3 × 10⁶ | 1.1 × 10⁵ |

The composition tested according to the invention exhibits good maintenance of the viability of the live bacterial strain by virtue of the presence of linear-chain liposoluble and lipodispersed molecules in sufficient amount.

### Example 3: Comparative Studies with Addition of Two Linear-Chain Molecules According to the Invention

### Protocol:

A composition 1 is produced from the same initial mixture in powder form comprising 50% by weight of the L. crispatus strain and 50% by weight of maltodextrin, which is dispersed at 1% (w/w) in a branched-chain ester, in this instance a capric/caprylic triglyceride, in sufficient amount to make the composition up to 100% (w/w).

A following composition 2 is produced from the same initial mixture in powder form comprising 50% by weight of the L. crispatus strain and 50% by weight of maltodextrin:

| | % (w/w) with |
|---|---|
| | respect to the |
| | final composition |
| Phase A: | |
| Caprylic/capric triglyceride | 39.08 |
| Coco-caprylate/caprate mixture | 20 |
| Vegetable oil | 20 |
| Linoleic acid | 20 |
| Tocopherol | 0.5 |
| Phase B: | |
| L. crispatus/maltodextrin mixture in a ratio of 1:1 | 0.42 |

The ingredients of phase A are mixed together at ambient temperature and then phase B is added with gentle stirring. The composition 2 contains 40% of linear-chain liposoluble and lipodispersed molecules according to the present invention.

The viability of the probiotic bacterial strain is monitored in the same way as in Example 2 (cf. [Table 2]).

### Results:

TABLE 2

| In CFU/g | T0 | T1 month | T2 months | T3 months | T5 months | T10 months | T12 months |
|---|---|---|---|---|---|---|---|
| Composition | 2 × 10⁴ | 0 | 0 | 0 | 0 | 0 | 0 |
| 1 at 4° C. | | | | | | | |
| Composition | 7.3 × 10⁶ | 9.4 × 10⁶ | 2.7 × 10⁶ | 3.3 × 10⁶ | 2.4 × 10⁶ | 2.6 × 10⁶ | 3.6 × 10⁶ |
| 2 at 4° C. | | | | | | | |
| Composition 2 | | 4.1 × 10⁶ | 1.6 × 10⁶ | 1.7 × 10⁶ | 8.5 × 10⁵ | 1.0 × 10⁶ | 2.1 × 10⁵ |
| at Tambient | | | | | | | |

After one day of storage at 4° C. in the composition 1 comprising a capric/caprylic triglyceride, live bacteria are no longer measured. The addition of an amount of 40% by weight, with respect to the total weight of the composition, of linear-chain liposoluble and lipodispersed molecules according to the present invention makes it possible to recognize the viability of the live probiotic bacterial strain.

### Example 4: Comparative Studies with Addition of Three Linear-Chain Molecules According to the Invention

A composition 3 is produced from the same initial mixture in powder form comprising 50% by weight of the L. crispatus strain and 50% by weight of maltodextrin, which is dispersed at 1% (w/w) in a fatty substance composed of a branched fatty chain of alcohol type, in this instance octyldodecanol, in sufficient amount to make the composition up to 100% (w/w).

A following composition 4 is produced from the same initial mixture in powder form comprising 50% by weight of the L. crispatus strain and 50% by weight of maltodextrin:

| | % (w/w) with |
|---|---|
| | respect to the |
| | final composition |
| Phase A: | |
| Dicaprylyl carbonate | 11.95 |
| Vegetable oil | 10 |
| Coco-caprylate/caprate mixture (Cetiol C5C) | 25 |
| Caprylic/capric triglyceride | 20 |
| Octyldodecanol | 25 |
| Tocopherol | 0.5 |
| Undecane and tridecane mixture (Cetiol ® Ultimate) | 5.00 |
| Vegetable oil of Argania spinosa nut (active | 2.50 |
| ingredient) | |
| Phase B: | |
| L. crispatus/maltodextrin mixture in a ratio of 1:1 | 0.05 |

The ingredients of phase A are mixed together at ambient temperature and then phase B is added with gentle stirring. The composition 4 contains 42% (w/w) of linear-chain liposoluble and lipodispersed molecules according to the present invention (dicaprylyl carbonate, coco-caprylate/caprate, and undecane and tridecane mixture).

The viability of the probiotic bacterial strain is monitored in the same way as in Example 2 (cf. [Table 3]).

### Results:

TABLE 3

| In | | T1 | T2 | T3 | T5 | T10 | T12 | T16 |
|---|---|---|---|---|---|---|---|---|
| CFU/g | T0 | month | months | months | months | months | months | months |
| Composition | 3.4 × 10⁴ | 1.8 × 10⁴ | 0 | 0 | 0 | 0 | 0 | 0 |
| 3 at 4° C. | | | | | | | | |
| Composition | 7.9 × 10⁶ | 4.1 × 10⁶ | 4.5 × 10⁶ | 4.8 × 10⁶ | 5.4 × 10⁶ | 2.0 × 10⁶ | 2.4 × 10⁶ | 7.4 × 10⁵ |
| 4 at 4° C. | | | | | | | | |
| Composition | | 7 × 10⁶ | 1.5 × 10⁶ | 2.7 × 10⁶ | 4.9 × 10⁶ | 1.4 × 10⁶ | 5.5 × 10⁵ | 4.3 × 10⁵ |
| 4 at | | | | | | | | |
| Tambient | | | | | | | | |

After two months of storage at 4° C. in the composition 3 comprising octyldodecanol, live bacteria are no longer measured. The addition of an amount of 42% by weight, with respect to the total weight of the composition, of linear-chain liposoluble and lipodispersed molecules according to the present invention makes it possible to maintain the viability of the live probiotic bacterial strain.

### Example 5: Comparative Studies with Addition of Four Linear-Chain Molecules According to the Invention

A following composition 5 is produced from the same initial mixture in powder form comprising 50% by weight of the L. crispatus strain and 50% by weight of maltodextrin:

| | % (w/w) with |
|---|---|
| | respect to the |
| | final composition |
| Phase A: | |
| Dicaprylyl carbonate | 13 |
| Coco-caprylate/caprate mixture (Cetiol C5C) | 13 |
| Shea butter | 4 |
| Cocoglycerides | 12 |
| Octydodecanol | 13.65 |
| Hydrogenated castor oil | 4 |
| Behenyl alcohol | 8 |
| Tocopherol | 0.5 |
| Microcrystalline cellulose | 20 |
| Phase B: | |
| Undecane and tridecane mixture (Cetiol ® Ultimate) | 10.00 |
| L. crispatus/maltodextrin mixture in a ratio of 1:1 | 0.05 |

The ingredients of phase A are mixed together while heating at 85° C. with stirring. When phase A is homogeneous, phase B is added at 65° C. with gentle stirring and then the combined mixture is left to cool to ambient temperature. The composition 5 contains 44% (w/w) of linear-chain liposoluble and lipodispersed molecules according to the present invention (dicaprylyl carbonate, coco-caprylate/caprate, undecane and tridecane mixture, and behenyl alcohol).

The viability of the probiotic bacterial strain is monitored in the same way as in Example 2 (cf. [Table 4]).

### Results:

TABLE 4

| Composition 5 | | | | | | | |
|---|---|---|---|---|---|---|---|
| according to the | | | | | | | |
| invention | | | | | | | |
| (CFU/g) | T0 | T1 month | T2 months | T3 months | T4 months | T5 months | T6 months |
| T at 4° C. | 7.9 × 10⁵ | 4.53 × 10⁵ | 2.53 × 10⁵ | 4.28 × 10⁵ | 3.59 × 10⁵ | 3.88 × 10⁵ | 3.7 × 10⁵ |
| Tambient | | 6.31 × 10⁵ | 2.81 × 10⁵ | 1.18 × 10⁵ | 1.65 × 10⁵ | 1.21 × 10⁵ | 6.6 × 10⁴ |

After two months of storage at 4° C. in the composition 3 comprising octyldodecanol, live bacteria are no longer measured. The addition of an amount of 44% by weight, with respect to the total weight of the composition, of linear-chain liposoluble and lipodispersed molecules according to the present invention makes it possible to maintain the viability of the live probiotic bacterial strain.

### Example 6: Comparative Studies with a Content of at Least 40% of Linear-Chain Molecules According to the Invention

### a) Formulation in a Branched-Chain Molecule, Squalane.

A composition is produced from the same initial mixture in powder form comprising 50% by weight of the L. crispatus strain and 50% by weight of maltodextrin, to which is added squalane at 1% by weight, with respect to the total weight of the composition (squalane+L. crispatus/maltodextrin mixture in a ratio of 1:1).

Squalane is a branched-chain molecule of formula C30H62.

The viability of the probiotic bacterial strain is monitored in the same way as in Example 2 at 4° C. (cf. [Table 5]).

### Results:

TABLE 5

| Composition | | | | |
|---|---|---|---|---|
| according to the | | | | |
| invention (CFU/g) | T0 | T1month | T2months | T3months |
| T at 4° C. | 2.17 × 10⁵ | 2 × 10⁵ | 8.5 × 10³ | 0 |

After two months of storage, the viability of the live bacterial strain has decreased by more than 2 log units and, at three months, live bacteria are no longer measured in the composition. These results make it possible to predict that the viability of the live bacterial strain at ambient temperature would be lost at least as rapidly, indeed even more rapidly.

b) Formulation in the Presence of at Least 30%/of Linear-Chain Molecules According to the Invention

A following composition 6 is produced from the same initial mixture in powder form comprising 50% by weight of the L. crispatus strain and 50% by weight of maltodextrin:

| | % (w/w) with |
|---|---|
| | respect to the |
| | final composition |
| Phase A: | |
| Shea butter | 46.45 |
| Rapeseed oil | 20.00 |
| Coco-caprylate/caprate mixture (Cetiol C5) | 20.00 |
| Passiflora incarnata seed oil | 5.00 |
| Myristyl myristate | 3.00 |
| Beeswax | 2.00 |
| Squalane | 3.00 |
| Tocopherol | 0.5 |
| Phase B: | |
| L. crispatus/maltodextrin mixture in a ratio of 1:1 | 0.05 |

The ingredients of phase A are mixed together while heating at 75° C. with stirring. When phase A is homogeneous, phase B is added at 65° C. with gentle stirring and then the combined mixture is left to cool to ambient temperature. The composition 6 contains at least 40% (w/w) of linear-chain liposoluble and lipodispersed molecules according to the present invention (coco-caprylate/caprate at 20%) and of other linear-chain liposoluble and lipodispersed molecules (rapeseed oil which contains 80% oleic acid, Passiflora incarnata seed oil, which contains at least 70% oleic acid, 7% linoleic acid, 1.4% behenic acid, 11% palmitic acid and 5% stearic acid, myristyl myristate and beeswax, which contains myricyl palmitate and cerotic acid, otherwise denoted hexacosanoic acid).

The viability of the probiotic bacterial strain is monitored in the same way as in Example 2 (cf. [Table 6]).

TABLE 6

| Composition 6 | | | | | | |
|---|---|---|---|---|---|---|
| according to the | | | | | | |
| invention (CFU/g) | T0 | T1 month | T2 months | T3 months | T4 months | T6 months |
| T at 4° C. | 8.7 × 10⁶ | 1.4 × 10⁷ | 1.1 × 10⁷ | 1.7 × 10⁷ | 6.76 × 10⁶ | 5.4 × 10⁶ |
| Tambient | 8.7 × 10⁶ | 1.7 × 10⁶ | 2.7 × 10⁵ | 1.3 × 10⁵ | 6.4 × 10³ | 4.4 × 10³ |

After three months of storage at 4° C. in the composition 6) comprising squalane, live bacteria are no longer measured. The addition of an amount of 40% by weight, with respect to the total weight of the composition, of linear-chain liposoluble and lipodispersed molecules according to the present invention and 10% by weight, with respect to the total weight of the composition, of other linear-chain liposoluble and lipodispersed molecules makes it possible to maintain the viability of the live probiotic bacterial strain. On the other hand, the presence of more than 40% of branched-chain liposoluble and lipodispersed molecules (shea butter+squalane) results in a decrease of 2 log units starting from 3 months, which reflects a loss of the viability at ambient temperature.

1.-24. (canceled)

25. A cosmetic and/or dermatological composition comprising:
at least one live probiotic bacterial strain,
at least one linear-chain lipodispersible and liposoluble molecule chosen from dicaprylyl carbonate; coco-caprylate, optionally combined with coco-caprate; undecane and/or tridecane; linoleic acid and/or oleic acid, and/or their linear-chain esters and/or oleyl alcohol; behenyl alcohol and/or its acid and ester derivatives; and/or their mixtures,
and optionally one or more other linear-chain lipodispersible and liposoluble molecules,
in which the total content of the linear-chain lipodispersible and liposoluble molecules is at least 40% by weight, with respect to the total weight of the composition.

26. The cosmetic and/or dermatological composition as claimed in claim 25, in which the other linear-chain lipodispersible and liposoluble molecule is chosen from:
alkane molecules of chemical formula CₙH₂ₙ₊₂ in which n denotes a value ranging from 6 to 40,
ester molecules of chemical formula CH₃-CₓH₂ₓ—COO—C_{y}H_{2y}—CH₃ in which each of the x and y indices independently denotes a value ranging from 5 to 19,
acid molecules of chemical formula CH₃-CₓH₂ₓ—C(O)OH in which x denotes a value ranging from 5 to 24, preferably from 5 to 21, such as cerotic acid,
alcohol molecules of chemical formula CH₃-CₓH₂ₓ—OH in which x denotes a value ranging from 5 to 21,
and any one of their mixtures.

27. The cosmetic and/or dermatological composition as claimed in claim 25, in which the linear-chain lipodispersible and liposoluble molecule chosen from dicaprylyl carbonate; coco-caprylate, optionally combined with coco-caprate, preferentially a mixture of coco-caprylate and of coco-caprate; undecane and/or tridecane; linoleic acid and/or oleic acid, and/or their linear-chain esters and/or oleyl alcohol; behenyl alcohol and/or its acid and ester derivatives, in particular behenic acid, or docosanoate or behenate esters; and/or their mixtures, is present at a content of at least 8%, with respect to the total weight of the composition.

28. The cosmetic and/or dermatological composition as claimed in claim 25, in which the linear-chain lipodispersible and liposoluble molecule is dicaprylyl carbonate.

29. The cosmetic and/or dermatological composition as claimed in claim 25, in which the linear-chain lipodispersible and liposoluble molecule chosen is coco-caprylate, optionally combined with coco-caprate.

30. The cosmetic and/or dermatological composition as claimed in claim 25, in which the linear-chain lipodispersible and liposoluble molecule chosen is dicaprylyl carbonate.

31. The cosmetic and/or dermatological composition as claimed in claim 25, in which the linear-chain lipodispersible and liposoluble molecule chosen is undecane and/or tridecane.

32. The cosmetic and/or dermatological composition as claimed in claim 25, in which the linear-chain lipodispersible and liposoluble molecule chosen is linoleic acid and/or oleic acid, and/or isostearyl linoleate and/or oleyl alcohol.

33. The cosmetic and/or dermatological composition as claimed in claim 25, in which the linear-chain lipodispersible and liposoluble molecule chosen is behenyl alcohol and/or its acid and ester derivatives.

34. The cosmetic and/or dermatological composition as claimed in claim 25, in which the linear-chain lipodispersible and liposoluble molecule chosen from dicaprylyl carbonate; coco-caprylate, optionally combined with coco-caprate; undecane and/or tridecane; linoleic acid and/or oleic acid, and/or their linear-chain esters and/or oleyl alcohol; behenyl alcohol and/or its acid and ester derivatives; and/or their mixtures, is present at a content of at least 40%, with respect to the total weight of the composition.

35. The composition as claimed in claim 25, in which the composition comprises at least two linear-chain lipodispersible and liposoluble molecules chosen from dicaprylyl carbonate; coco-caprylate, optionally combined with coco-caprate; undecane and/or tridecane; linoleic acid and/or oleic acid, and/or their linear-chain esters and/or oleyl alcohol; behenyl alcohol and/or its acid and ester derivatives.

36. The composition as claimed in claim 25, in which the composition additionally comprises a branched-chain liposoluble and lipodispersible molecule chosen from octyldodecanol, propylheptyl caprylate, a mixture of caprylic and capric triglycerides, hydrogenated castor oil, shea butter, coconut oil and/or cocoglycerides.

37. The composition as claimed in claim 25, in which the live probiotic bacterial strain is chosen from Lactobacillus crispatus, Corynebacterium accolens, Sphingomonas glacialis, Streptococcus salivarus and their mixtures.

38. The composition as claimed in claim 25, in which the live bacterial strain is formulated from powder obtained by lyophilization on a maltodextrin support.

39. The composition as claimed in claim 25, in which the live bacterial strain is at a content of 10³ to 10¹⁵ CFU (colony-forming unit) per gram of composition.

40. The composition as claimed in claim 25, in which the live bacterial strain is at a content of 0.01% to 3% by weight, with respect to the total weight of the composition.

41. The composition as claimed in claim 25, in which the water activity (Aw) in the composition is less than 0.6.

42. The composition as claimed in claim 25, characterized in that it is in the form of an oily suspension or solution, an oily cream or gel, a milk, an emulsion of water-in-oil or multiple or silicone type, a mask, a serum, a lotion, a solid soap, a shampoo in solid form, foaming products in solid form, a dermatological bar, an ointment, a mousse, a patch, an anhydrous product.

43. The non-therapeutic use of a cosmetic composition as defined in claim 25, for preventing and/or reducing unaesthetic and/or uncomfortable manifestations of healthy skin, healthy superficial body growths and/or healthy mucous membranes, superficial body growths and/or mucous membranes, of fragile or weakened skin, superficial body growths and/or mucous membranes, of dry skin, superficial body growths and/or mucous membranes, and/or of skin, superficial body growths and/or mucous membranes having an atopic tendency.

44. The non-therapeutic use of a cosmetic composition as defined in claim 25, as moisturizing and/or soothing agent for healthy skin, healthy superficial body growths and/or healthy mucous membranes.

45. The non-therapeutic use of the cosmetic composition as defined in claim 25 by topical application, to an area of healthy skin, mucous membrane and/or superficial body growth chosen from the legs; the feet; the armpits; the hands; the thighs; the hips; the buttocks; the waist; the crotch; the groin; the stomach; the neckline; the neck; the arms; the torso; the back; the face; the scalp; the hair; the nails; and/or the labial mucous membrane.

46. The dermatological composition as defined in claim 25, for its use as anti-inflammatory agent or antibacterial agent and/or in the treatment and/or the prevention of infections of the skin and/or mucous membranes and/or superficial body growths caused by a pathogenic microorganism, eczema, seborrhoeic or atopic dermatitis, acne, and cutaneous or mucosal inflammation caused by a pathogenic microorganism, such as a pathogenic yeast or bacterium, such as S. aureus, mycosis and/or erythema, and/or in the prevention and/or the pharmaceutical treatment, of pathologies related to reactive and/or atopic skin and/or mucous membranes.

47. The use of at least one linear-chain lipodispersible and liposoluble molecule chosen from dicaprylyl carbonate; coco-caprylate, optionally combined with coco-caprate; undecane and/or tridecane; linoleic acid and/or oleic acid, and/or their linear-chain esters and/or oleyl alcohol; behenyl alcohol and/or its acid and ester derivatives; and/or their mixtures, and optionally one or more other linear-chain lipodispersible and liposoluble molecules, for maintaining the viability of a live probiotic bacterial strain, in which the total sum by weight of the linear-chain lipodispersible and liposoluble molecules is at least 40% by weight, with respect to the total weight of a cosmetic or dermatological composition.
